(19) United States
(12) Patent Application Publication
(10) Pub. No.: US 2025/0318872 A1
(43) Pub. Date: Oct. 16, 2025

(54) LASER RESONATOR AND OPTICAL PUMPING SYSTEM FOR MEDICAL LASER SYSTEMS

(71) Applicant: LUMENIS LTD., Yokneam Illit (IL)

(72) Inventor: Arkady Khachaturov, Kiryat Yam (IL)

(73) Assignee: LUMENIS LTD., Yokneam Illit (IL)

(21) Appl. No.: 19/173,093

(22) Filed: Apr. 8, 2025

Related U.S. Application Data
(60) Provisional application No. 63/634,307, filed on Apr. 15, 2024.

(51) Int. Cl. A61B 18/20

(52) U.S. Cl. CPC A61B 18/20; A61B 2018/20553; A61B 2018/205547

(57) ABSTRACT

An optical resonator for a medical laser system is provided. The optical resonator comprises a lasing medium, an optical pump, and a high-reflective (HR) and an optical coupling (OC) reflector. The HR and OC reflectors are disposed symmetrically relative to the lasing medium and the HR mirror, the OC mirror, or both the HR mirror and the OC mirror are flat mirrors. The optical resonator also includes a doped insert in which the lasing medium is disposed.

### CROSS-REFERENCE TO RELATED APPLICATIONS

This application claims the benefit of U.S. Provisional Application No. 63/634,307, filed Apr. 15, 2024, the disclosure of which is incorporated herein by reference in its entirety.

### TECHNICAL FIELD

The present disclosure generally relates to surgical laser system. Particularly, but not exclusively, the present disclosure relates to surgical laser systems used in lithotripsy procedures.

### BACKGROUND

Medical lasers are used in a variety of procedures. Among several of the procedures, laser energy is directed towards a target using a fiber as a conduit for the laser energy. One such procedure, to address renal calculi (e.g., kidney stones) is ureteral endoscopy, or lithotripsy. An endoscopic probe, with a camera or other sensor, is inserted into the patient's urinary tract to locate the calculi for removal. In endoscopic lithotripsy, the probe also includes an optical fiber, which conducts a laser beam to break up, disintegrate, or otherwise irradiate the calculi as they are found.

One of main requirements for the lasers systems used in lithotripsy is their ability to generate pulses at high frequency (e.g., repetition rate, or the like). One possible way to increase the frequency is to implement multiple individual laser generating sub-systems, or “bricks”, within the laser system, where the bricks are configured to sequentially generate laser pulses. These laser pulses are optically combined into a composite or single output laser beam, which includes all the generated laser pulses. For example, a laser system having four (4) bricks could be implemented to generate an output laser beam having repetition rates up to four (4) times higher than that of each individual brick. Although laser systems with multiple bricks are “theoretically” scalable, in practice the increased cost and size resulting from adding multiple bricks reduces the practicality of such systems.

Thus, there is a need to improve the repetition rate or frequency of laser generating system used in medical laser systems.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a laser generating component, or “brick” that can be integrated into a medical laser console and used to supply all or a portion of the laser energy of the console. In general, the laser brick component includes a lasing medium, reflectors, optical pumping components, cooling components, and/or control electronics. In general, the present disclosure provides an optical brick where the reflectors (e.g., HR and OC reflectors) are symmetrically relative to the lasing medium. Furthermore, the optical brick comprises an insert having a doping and in which the lasing medium is disposed.

With some embodiments, the disclosure can be implemented as an optical resonator for a medical laser system. The optical resonator can comprise a housing; an optical pump source disposed in the housing; an insert disposed in the housing; a lasing medium disposed in the insert; a high-reflectivity (HR) mirror disposed outside the housing a first distance away from a first end of the lasing medium; and an output coupler (OC) mirror disposed outside the housing a second distance away from a second end of the lasing medium, wherein the first end of the lasing medium is opposite the second end of the lasing medium, and wherein the first distance is substantially equal to the second distance.

In further embodiments of the optical resonator, the HR mirror, the OC mirror, or both the HR mirror and the OC mirror are flat mirrors.

In further embodiments of the optical resonator, the insert comprises Samarium (Sm).

In further embodiments of the optical resonator, the insert is Sm-doped Fused Silica.

In further embodiments of the optical resonator, the first distance and the second distance are greater than or equal to 0 millimeters (mm) and less than or equal to 120 mm.

In further embodiments of the optical resonator, the lasing medium is a Ho:YAG lasing rod or a CTH:YAG lasing rod.

In further embodiments of the optical resonator, the optical pump source is configured to energize at a frequency of greater than or equal to 20 Hertz (Hz).

In further embodiments of the optical resonator, the optical pump source is configured to energize at a frequency of greater than 20 Hz and less than or equal to 45 Hz.

With some embodiments, the disclosure can be implemented as a method for energizing an optical resonator for a medical laser system. The method can comprise receiving an indication to initiate lasing by the optical resonator; activating, for a selected time period at a preselected repetition rate and output energy, an optical pump source of the optical resonator; sending, after a selected delay, a control signal to a shutter of the medical laser system to cause the shutter to open; and activating the optical pump source at a repetition rate and output energy specified for a treatment, wherein the preselected repetition rate is greater than or equal to 85 percent of a maximum repetition rate of the optical pump source, and wherein the preselected output energy is substantially equal to a lasing threshold of a laser medium of the optical resonator.

In further embodiments of the method, the selected delay equals the selected time period.

In further embodiments of the method, the selected delay and the selected time period are less than or equal to 200 milliseconds (ms).

In further embodiments of the method, the maximum repetition rate of the optical pump source is greater than or equal to 20 Hertz (Hz).

In further embodiments of the method, the maximum repetition rate of the optical pump source is greater than 20 Hz and less than or equal to 45 Hz.

In further embodiments of the method, the optical resonator comprises a housing, the optical pump source disposed in the housing; an insert disposed in the housing; the lasing medium disposed in the insert; a high-reflectivity (HR) mirror disposed outside the housing a first distance away from a first end of the lasing medium; and an output coupler (OC) mirror disposed outside the housing a second distance away from a second end of the lasing medium, wherein the first end of the lasing medium is opposite the second end of the lasing medium, and wherein the first distance is substantially equal to the second distance.

In further embodiments of the method, the HR mirror, the OC mirror, or both the HR mirror and the OC mirror are flat mirrors.

In further embodiments of the method, the insert comprises Samarium (Sm).

In further embodiments of the method, the insert is Sm-doped Fused Silica.

In further embodiments of the method, the lasing medium is a Ho:YAG lasing rod or a CTH:YAG lasing rod.

With some embodiments, the disclosure can be implemented as a medical laser system. The medical laser system can comprise at least one optical resonator, each of the at least one optical resonators comprising: a housing, an optical pump source disposed in the housing, an insert disposed in the housing, a lasing medium disposed in the insert, a high-reflectivity (HR) mirror disposed outside the housing a first distance away from a first end of the lasing medium, and an output coupler (OC) mirror disposed outside the housing a second distance away from a second end of the lasing medium; a shutter; at least one mirror configured to reflect an output emission from the at least one optical resonator to the shutter; and a coupling assembly comprising at least one lens configured to optically couple the output emission with a proximal end of an optical fiber, wherein the first end of the lasing medium is opposite the second end of the lasing medium, and wherein the first distance is substantially equal to the second distance.

In further embodiments of the medical laser system, the insert is Sm-doped Fused Silica.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates a medical laser system in accordance with at least one embodiment of the disclosure.

FIG. 1B illustrates an optical fiber of the medical laser system of FIG. 1A in greater detail.

FIG. 1C illustrates an optical deck of the medical laser system of FIG. 1A in greater detail.

FIG. 2 illustrates an optical resonator in accordance with at least one embodiment of the disclosure.

FIG. 3 illustrates a graph depicting operational aspects of the optical resonator of FIG. 2.

FIG. 4A illustrates another graph depicting operational aspects of the optical resonator of FIG. 2.

FIG. 4B illustrates a graph depicting operational aspects of a conventional optical resonator.

FIG. 5 illustrates a routine for warming up an optical resonator in accordance with at least one embodiment of the disclosure.

FIG. 6A illustrates yet another graph depicting operational aspects of the optical resonator of FIG. 2.

FIG. 6B illustrates still another graph depicting operational aspects of the optical resonator of FIG. 2.

FIG. 7 illustrates a computing device in accordance with at least one embodiment of the disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

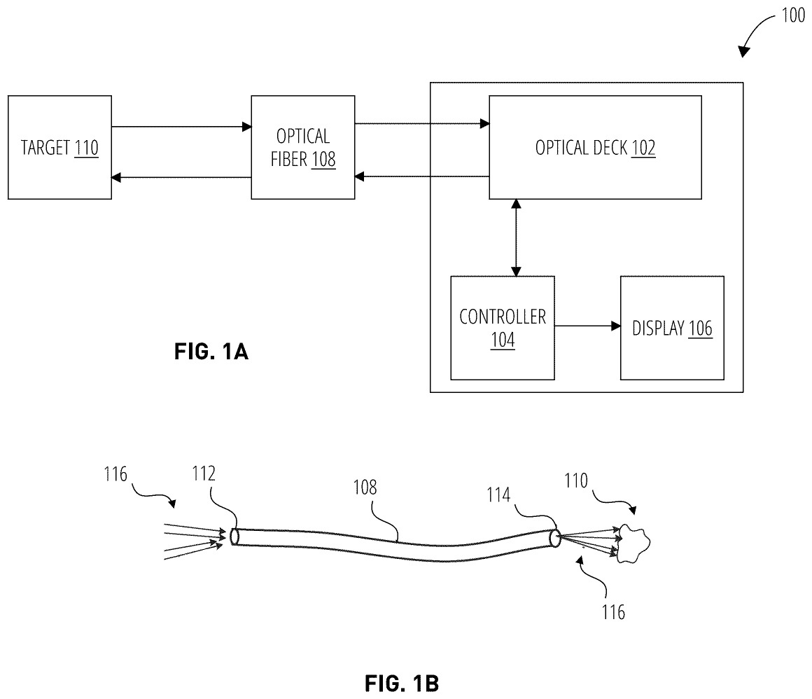

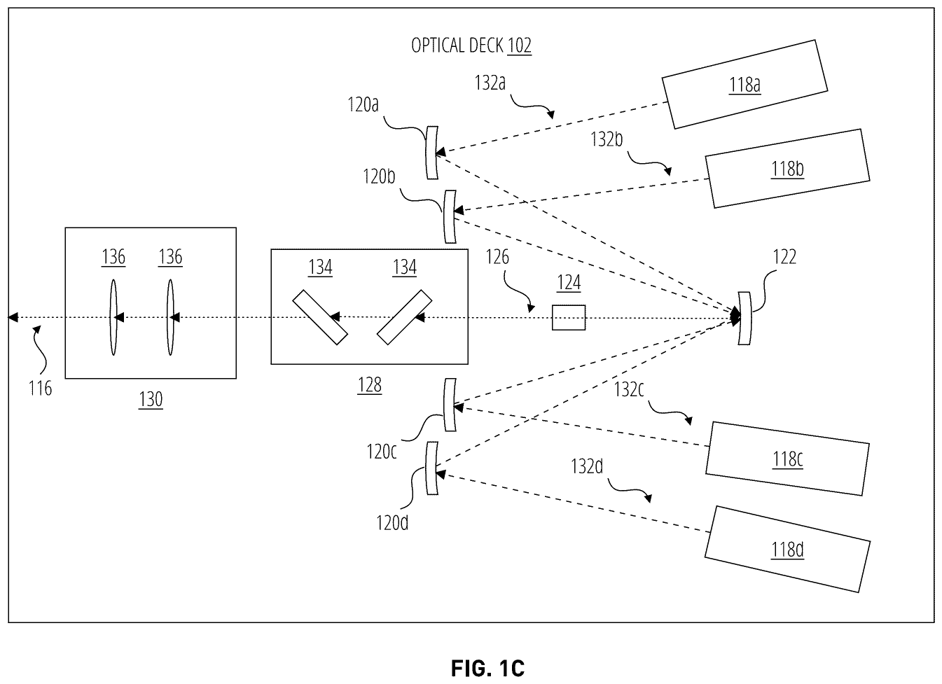

FIG. 1A, FIG. 1B, and FIG. 1C, show an exemplary medical laser system 100 for generating a pulsed laser beam. In general, the medical laser system 100 comprises an optical deck 102, a controller 104, and a display 106. The optical deck 102 is configured to be optically coupled to optical fiber 108. During operation, medical laser system 100 can generate a pulsed laser beam and convey the pulsed laser beam to a target 110 via the optical fiber 108. In some examples, the target 110 may be a tissue, a stone, a tumor, a cyst, and the like. The target 110 may be located within a subject (e.g., a human, an animal, or the like). The target 110 may be treated (e.g., ablated, disintegrated, dusted, shattered, or the like).

The controller 104 may be associated with and/or communicatively coupled the display 106. In general, controller 104 can include processing circuitry (e.g., a processor unit, a microcontroller, or the like) and computer-readable memory storing instructions that when executed by the processing circuitry cause the controller 104 to control components or the optical deck 102 to generate a laser beam as outlined herein. Parameters and/or characteristics of the generated laser beam can be displayed on display 106.

As depicted more fully in FIG. 1B, the optical fiber 108 comprises a proximal end 112 and a distal end 114. The proximal end 112 is the end of the optical fiber 108 through which light beams enter while the distal end 114 is the end of the optical fiber 108 through which light beams are emitted and via which light beams can be directed onto the target 110. For example, this figure depicts incident light 116 entering the optical fiber 108 at the proximal end 112, propagating through the length of the optical fiber 108, exiting the optical fiber 108 at the distal end 114, and being incident on the target 110 from the distal end 114 of the optical fiber 108.

In general, the optical deck 102 can be configured to generate a pulsed laser beam (not shown). As depicted more fully in FIG. 1C, the optical deck can include several bricks, or optical resonators. For example, optical deck 102 is depicted including optical resonators 118a, 118b, 118c, and 118d. It is noted that optical deck 102 could include any integer number of optical resonators greater than 2. The optical deck 102 is depicted including four (4) optical resonators for purposes of clarity of presentation only and not for limitation. Optical deck 102 further includes mirrors 120a, 120b, 120c, 120d, and 122, a shutter 124, a diagnostic assembly 128, and a coupling assembly 130.

In general, the optical resonators 118a, 118b, 118c, and 118d are each configured to generate pulsed laser beams 132a, 132b, 132c, and 132d, respectively. Pulsed laser beams 132a, 132b, 132c, and 132d can have a variety of pulse shapes, pulse widths, pulse delays, frequency, and/or magnitude. Further, the pulse waveform of each of pulsed laser beams 132a, 132b, 132c, and 132d need not be the same. With some embodiments, the pulsed laser beams 132a, 132b, 132c, and 132d can have a frequency greater than or equal to 20 Hertz (Hz), greater than 20 Hz, greater than or equal to 20 Hz and less than or equal to 45 Hz, greater than 20 Hz and less than or equal to 45 Hz, or substantially equal to 45 Hz.

Optical resonators 118a, 118b, 118c, and 118d can be any of a variety of laser light sources, such as for example, solid-state lasers, gas lasers, diode lasers, and fiber lasers. In a particular example, optical resonators 118a, 118b, 118c, and 118d can be based on a solid state laser medium, such as, for example, Holmium (Ho) based lasers (e.g., Ho:YAG, or the like). As another example, optical resonators 118a, 118b, 118c, and 118d can be based on a multiple doped laser medium, such as, for example, a triple doped medium (e.g., Chromium (Cr), Thulium (Tm), and Ho (e.g., CTH:YAG, or the like).

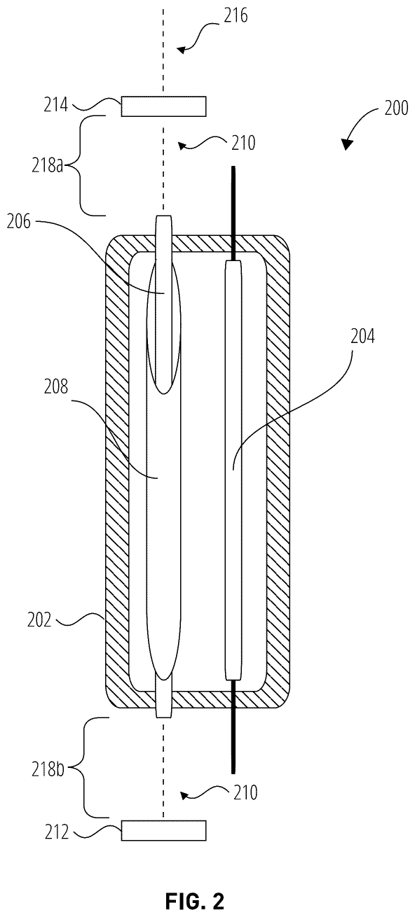

An example of an optical resonator (e.g., one of optical resonators 118a, 118b, 118c, or 118d) is depicted in more detail in FIG. 2. Optical resonators 118a, 118b, 118c, and 118d can be coupled to controller 104 where controller 104 is configured to cause Optical resonators optical resonator 118a, 118b, 118c, and 118d to generate pulsed laser beams 132a, 132b, 132c, and 132d having specific characteristics (e.g., frequency, power, etc.).

During operation, optical resonators 118a, 118b, 118c, and 118d can be configured to sequentially generate pulsed laser beams 132a, 132b, 132c, and 132d, which are optically aligned with respective mirrors 120a, 120b, 120c, and 120d. Mirrors 120a, 120b, 120c, and 120d reflect and/or redirect pulsed laser beams 132a, 132b, 132c, and 132d to mirror 122. Mirror 122 may be a galvanometer, circulator, a rotating mirror, or other optical element configured to combine and redirect each of pulsed laser beams 132a, 132b, 132c, and 132d towards shutter 124 as combined pulsed laser beam 126. Shutter 124 may be electronically coupled to controller 104 and configured to open or close to turn on or turn off output of the combined pulsed laser beam 126.

Diagnostic assembly 128 can include a variety of optical elements, such as, for example, beam splitters 134, diagnostic light sources (not shown), sensors (not shown), mirrors (not shown), etc. Diagnostic assembly 128 can be coupled to controller 104 and configured to provide diagnostic and/or measurement features to medical laser system 100. For example, controller 104 and diagnostic assembly 128 can include optical elements and be arranged to determine a distance between the target 110 and the distal end 114 of the optical fiber 108, identify the type or class of the target 110, or the like.

Coupling assembly 130 can include a variety of optical elements, such as, for example, lenses 136. In general, coupling assembly 130 can be configured to shape and/or reduce spherical aberrations in combined pulsed laser beam 126 and optical couple combined pulsed laser beam 126 to the proximal end 112 of the optical fiber 108.

As noted, optical deck 102 can include a variety of optical elements which may include, but are not limited to, one or more of light sources, polarizers, beam splitters, beam combiners, light detector, wavelength division multiplexers, collimators, circulators, etc., which are arranged and configured in various combinations as explained and evident from the present disclosure.

In many embodiments, laser light sources are configured to generate laser light beams, such as a low intensity aiming beam for the purpose of aiming the combined pulsed laser beam 126 at the target 110 and a high intensity treatment beam (e.g., combined pulsed laser beam 126) for treating the target 110, and/or light beams with varying characteristics (e.g., intensities, wavelengths, etcetera) based on the application. Each laser light source may be configured to generate laser light having different wavelengths, where each of the different wavelengths can have different water absorption coefficients. Additionally, laser light sources may be configured to generate polarized laser light or unpolarized/depolarized light.

Polarizers may include the optical components that act as an optical filter. For example, polarizers may be configured to allow light beams of a specific polarization to pass through, and to block the light beams of different polarizations. Therefore, when undefined light (or light beams of mixed polarity) is provided as input to a polarizer, the polarizer provides a well-defined single polarized light beam as an output.

Beam splitters may include the optical components used to split incident light at a designated ratio into two separate beams. Further, beam splitters may be arranged to manipulate light to be incident at a desired angle of incidence (AOI). Therefore, in many embodiments, a beam splitter can be primarily configured with two parameters, a ratio of separation and an AOI. The ratio of separation comprises the ratio of reflection to transmission (reflection/transmission (R/T) ratio) of the beam splitter.

Beam combiners may include partial reflectors that combine two or more wavelengths of light, such as by using the principle of transmission and reflection as explained above. In many embodiments, a beam combiner may be a combination of beam splitters and mirrors, which perform the functionality of combining light of two or more wavelengths.

Light detectors may include devices that detect and/or measure characteristics of light beams and encode the detected and/or measured characteristics in electrical signals. For example, light detectors may detect the specific type of light beams (as preconfigured), and convert the light energy associated with the detected light beams into electrical signals.

A collimator may include a device that narrows down light beams. To narrow down the light beam, a collimator may be configured to cause the directions of motion to become more aligned in a specific direction (for example, parallel rays), or to cause the spatial cross section of the beam to become smaller. In many embodiments, a collimator may be used to change diverging light from a point source into a parallel beam.

A circulator may include a multi-port optical device configured to receive and emit light via a predetermined sequence of the multiple ports. For example, a circulator may include a three (or four, or five, etc.) port optical device designed such that, light entering any one port exits from the next port. In one such example, light entering a first port may exit a second port, light entering the second port may exit a third port, and light entering the third port may exit the first port. Oftentimes circulators may be utilized to allow light beams to travel in only one direction.

FIG. 2 illustrates an example optical resonator 200, which can be implemented as any one of the optical resonators depicted and described herein, such as, for example, optical resonators 118a, 118b, 118c, and 118d. Optical resonator 200 includes a housing 202 in which is disposed an optical pump source 204, a laser rod 206 (or lasing medium), and an insert 208. It is noted that the laser rod 206 is disposed within the insert 208.

In some examples, optical pump source 204 can be a broadband pump source (e.g., a flash lamp, or the like). For example, in some embodiments, the optical pump source 204 can be a Xenon (Xe) based flash lamp. The optical pump source 204 can be coupled to controller 104 and configured to provide optical pump light to the laser rod 206. With some embodiments, although not shown, the optical pump source 204 can be powered by a capacitor bank. The optical pump light operates to supply energy to the laser rod 206 to cause atomic population inversion in the laser rod 206, thus ultimately achieving a stimulated emission 210 from the laser rod 206.

The optical resonator 200 further includes a mirror 212 and a mirror 214. In general, mirror 212 is a high-reflection (HR) mirror that is arranged and configured to focus the stimulated emission 210 back into the laser rod 206. Similarly, mirror 214 is arranged and configured to focus the stimulated emission 210 back into the laser rod 206. However, mirror 214 has a lower reflection coefficient than mirror 212, often referred to as an output coupler (OC). As such, during operation, when the stimulated emission 210 reaches a specific level of intensity, some, or all stimulated emission 210 passes through mirror 214 as a pulsed laser beam 216.

In some examples, mirror 212 and mirror 214 are flat mirrors, as opposed to concave as is conventionally used. Further, with some embodiments, the optical resonator 200 is designed and implemented to be symmetrical. Said differently, mirror 212 and mirror 214 are disposed equal distances away from the respective ends of laser rod 206. For example, distance 218a and distance 218b can be equal, or substantially equal.

In some examples, distances 218a and 218b can be zero, such as, when mirrors 212 and 214 are coated onto the ends of the laser rod 206. In some examples, the distances 218a and 218b can be as large as the distance at which the diameter of the diverging beam after reflection in the mirrors 212 and 214 becomes commensurate with the diameter of the laser rod 206. It is to be appreciates that the upper limit of the distances 218a and 218b further depends on the length of the laser rod 206. In some embodiments, the distances 218a and 218b can be greater than or equal to zero (0) millimeters (mm) and less than or equal to 100 mm, greater than or equal to 20 mm and less than or equal to 120 mm, greater than or equal to 20 mm and less than or equal to 100 mm, greater than or equal to 20 mm and less than or equal to 120 mm, greater than or equal to 40 mm and less than or equal to 80 mm, or equal to or substantially equal to 60 mm.

Use of flat mirrors for mirror 212 and mirror 214 arranged in a symmetrical configuration with laser rod 206 provides an increase in the maximum power at which the pulsed laser beam 216 can be generated. Due to the flat mirrors 212 and 214, the optical resonator 200 supports a larger number of radiation modes (e.g., rays propagating over a wider range of angles), thereby increasing the maximum power output. For example, an optical resonator 200 according to the present disclosure can be configured to generate pulsed laser beam 216 having 45 Hz and 60 Watts.

As noted, optical resonator 200 includes insert 208 disposed within housing 202. In general, the insert 208 can be a tube that is configured to filter and/or absorb some wavelengths of light (e.g., light in the ultra-violet (UV) spectrum, or the like). In some specific examples, the insert 208 is a Samarium (Sm) doped glass tube in which the laser rod 206 is disposed. For example, where the optical pump source 204 is a Xe flash lamp, an Sm-doped filter can be selected as the insert 208. As such, during operation, the Sm-doped insert 208 operates to absorb a portion of the heat generated by the optical pump source 204 and redistribute the heat between the insert 208 and the laser rod 206, thereby preventing the laser rod 206 from overheating, resulting in an increase in the possible maximum frequency. In such an example, where the optical pump source 204 is a Xe flash lamp, the Chromium (Cr)-ion absorption level of the laser rod 206 will be suppressed some degree based on the Sm-doped insert 208 spectrally filtrating, in the UV-region, the output of the light from the Xe-based optical pump source 204.

Optical resonator 200 can further include cooling components (not shown) coupled to housing 202 and/or component of housing 202, such as, insert 208. The cooling components can be configured to remove heat from the optical resonator 200 (e.g., via cooling liquid flow, cooling airflow, or the like). It is noted that the total amount of energy absorbed into the housing 202 will not change. However, the insert 208 provides reduction in overheating of the laser rod 206. That is, the laser rod 206 can operate as lower temperatures than conventionally possible as the insert 208 absorbs a portion of the heat generated by the optical pump source 204 and with which is removed via cooling components of the system.

An advantage of the optical resonator 200 shown in FIG. 2 results from the reduced working or operating temperature of the laser rod 206. For example, pumping of a CTH:YAG laser can start with excitation of the UV levels of Cr-ions, which is then transferred by non-radiative transitions to Tm-ions and then, after a cross-relaxation process, to Ho-ions. Non-radiative energy transfer occurs by means of phonons. As the excited ions increase in size and push on their neighbors, thermal motion is increased. As a result, at high powers CTH:YAG rods becomes very hot and lose efficiency requiring more powerful cooling and/or shorter repetition rates.

The present disclosure provides a reduced working temperature of a CTH:YAG rod, thereby resulting in increased frequencies and/or higher power output from a single resonator.

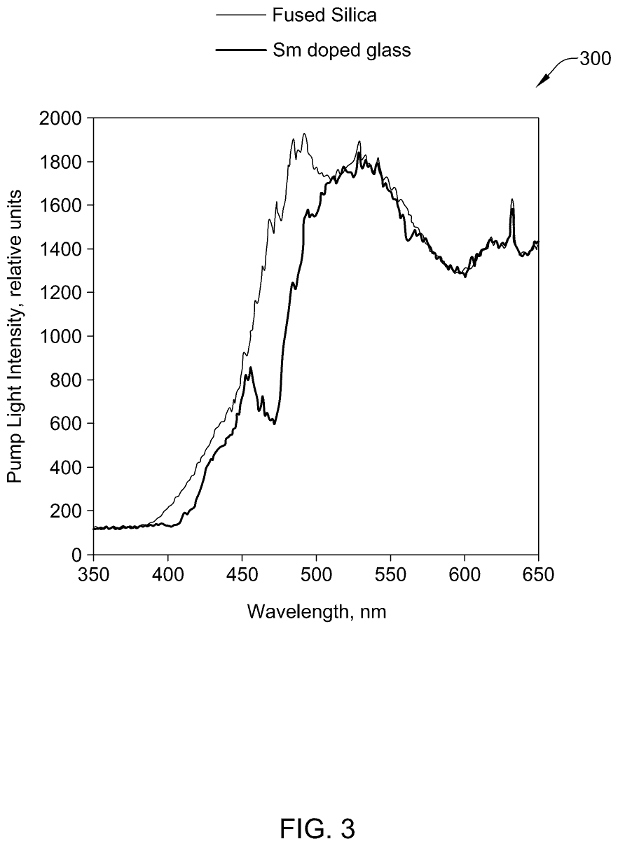

FIG. 3 illustrates a graph 300 showing intensity of the light from optical pump source 204 on the y axis versus the output wavelength on the x axis for both an Sm-doped insert 208 and a Fused Silica (e.g., glass or fused quartz) insert 208. As can be seen, the light intensity is slightly less at wavelengths between about 450 nanometers (nm) and 700 nm where an Sm-doped insert 208 is used. Accordingly, the Sm-doped insert 208 absorbs heat associated with the light from optical pump source 204 at these wavelengths.

It is noted, theoretically laser cavities implemented with flat mirrors (e.g., as depicted in FIG. 2) are non-stable. As such, in conventional systems concave mirrors are used to angle the stimulated emission 210 back into the laser rod 206. However, the present disclosure provides to use flat mirrors for mirror 212 and mirror 214. It will be appreciated that as the laser rod 206 absorbs energy (e.g., from optical pump source 204) the temperature of the laser rod 206 will increase. However, the temperature distribution is not uniform in the laser rod 206 and will typically be higher at the axis. As such, the laser rod 206 becomes a lens and will focus the stimulated emission 210 providing that use of flat mirrors for mirrors 212 and mirror 214 will be stable enough, thereby allowing practical operation of the optical resonator 200 at intense thermal regimes.

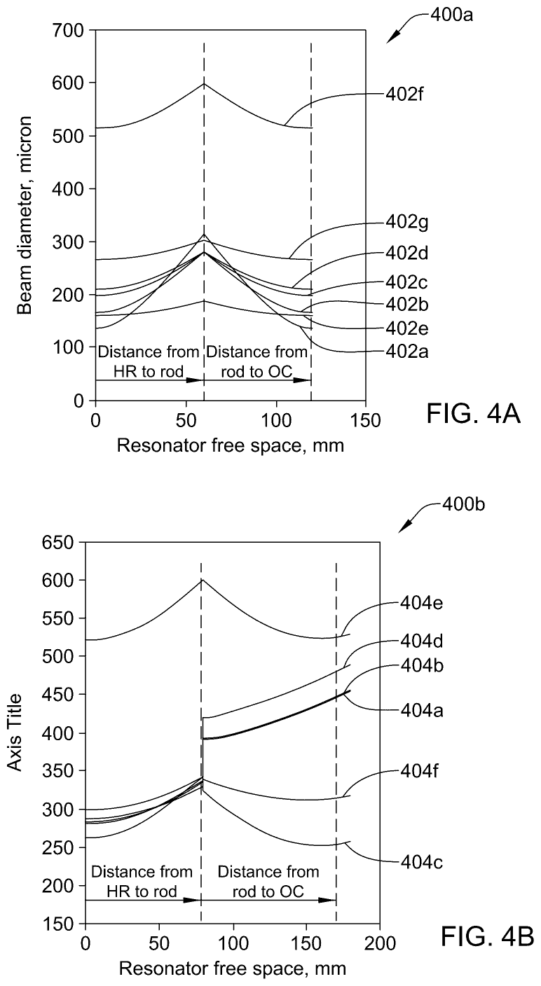

FIG. 4A and FIG. 4B illustrate graphs 400a and 400b, respectively. It is noted that the graphs were generated based on a simulation where the laser rod 206 was modeled as a nonlinear medium with quadratic dependence of refractive index over the rod radius.

FIG. 4A illustrates graph 400a showing the beam diameter (in microns) from pulsed laser beam 216 on the y-axis versus distance 218a (e.g., “distance from rod to OC”) and the distance 218b (e.g., “distance from HR to rod”) in millimeters (mm) on the x-axis. As depicted in this figure, the distances 218a and 218b are equal. The beam diameter versus distance depicted in FIG. 4A for several different focal lengths (FLs) 402. As used herein, the FL is the measure of the thermal lensing effect of the laser rod 206. Thus, as used herein, FL 402 is the FL of the effective lens formed by the non-uniform heated laser rod 206. For example, FL 402a through 402g are depicted where FL 402a equals 40 mm, FL 402b equals 45 mm, FL 402c equals 55 mm, FL 402d equals 60 mm, FL 402e equals 85 mm, FL 402f equals 90 mm, and FL 402g equals 100 mm.

FIG. 4B illustrates graph 400b showing the beam diameter (in microns) on the y-axis where the beam diameter is from a pulsed laser beam generated using an optical brick with concave HR and OC mirrors disposed non-symmetrically away from the rod versus distance 218a (e.g., “distance from rod to OC”) and the distance 218b (e.g., “distance from HR to rod”) in millimeters (mm) on the x-axis. As depicted in this figure, the distances 218a and 218b are not equal. The beam diameter versus distance for several different FLs 404 are also depicted. For example, FL 404a through FL 404f are depicted where FL 404a equals 300 mm, FL 404b equals 250 mm, FL 404c equals 100 mm, FL 404d equals 200 mm, FL 404e equals 135 mm, and FL 404f equals 150 mm.

It is to be appreciated from graphs 400a and 400b shown in FIG. 4A and FIG. 4B that for a given regime, symmetrical resonators (e.g., where distance 218a and distance 218b are equal) supports generating an output pulsed laser beam 216 in which the beam diameter is uniform over the rod length. As such, pump energy usage is more effective and efficient as more energy accumulated in the rod volume is going to the generation of the pulsed laser beam 216.

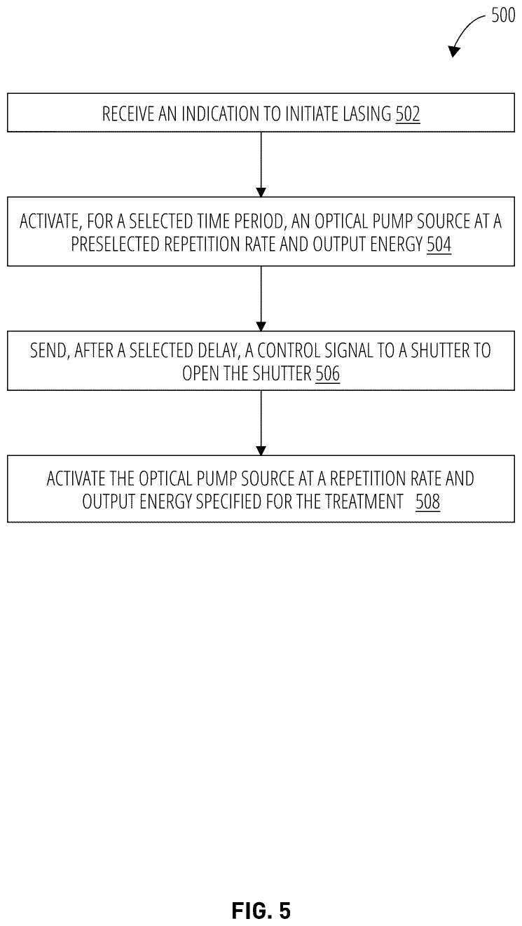

FIG. 5 illustrates a routine 500, which can be implemented by a laser system to provide a pulsed laser beam as described herein. In some embodiments, the medical laser system 100 can be configured to implement the routine 500. For example, controller 104 can be arranged (e.g., include instructions stored in memory, which are executable by processing circuitry) to carry out the routine 500.

In general, routine 500 can be implemented to “warm up” the laser rod 206. It is to be appreciated that as the working or operating temperature of the laser rod 206 is reduced versus conventional bricks, due in part to the insert 208, upon initiation of lasing first pulses that are lased when the rod temperature is significantly lower than that of the steady state may exceed acceptable limits. For example, the first few (e.g., tens, or the like) number of pulses of the pulsed laser beam 216 may exceed the specified power by up to three (3) times. It is to be appreciated that this can lead to damage of the laser rod 206. This may be particularly true where the pulses are generated at close to the maximum frequency. Accordingly, routine 500 is provided to warm up the laser rod 206 and reduce likelihood of damage to the laser rod 206 and/or the patient.

Routine 500 can begin at block 502. At block 502 “receive an indication to initiate lasing” controller 104 can receive an indication to initiate lasing. For example, medical laser system 100 can be provided with an input device (e.g., foot pedal, or the like) which is communicatively and/or electrically coupled to controller 104. Upon actuation of the input device (e.g., depression of the foot pedal, or the like) controller 104 can receive an indication (e.g., control signal, or the like) from the input device, where the control signal indicates that the medical laser system 100 should generate a pulsed laser beams 216, which can be communicated to optical fiber 108 as incident light 116. For example, processing circuitry of controller 104 can receive a control signal from the input device (e.g., foot pedal) responsive to activation of the input device.

Continuing to block 504 “activate, for a selected time period, an optical pump source at a preselected repetition rate and output energy” the optical pump source 204 can be activated, for a selected time period, at a preselected, or predefined, repetition rate and output energy. For example, processing circuitry of controller 104 can execute instructions stored in memory to cause controller 104 to send a control signal to optical pump source 204 to cause optical pump source 204 to energize at a preselected repetition rate and power for several repetitions. In some embodiments, controller 104 can send a control signal to optical pump source 204 to cause optical pump source 204 to energize at the maximum repetition rate and at a power just below the lasing threshold of the laser rod 206 for a time period of 150 milliseconds (ms), a time period of greater than or equal to 150 ms and less than or equal to 300 ms, or a time period of 200 ms.

With some examples, the preselected repetition rate can be within a threshold level (e.g., 99%, 95%, 90%, 85%, or the like) of the maximum repetition rate of the optical pump source. Similarly, the preselected output power can be within a threshold level (e.g., 99%, 95%, 90%, 85%, or the like) of the lasing threshold of the laser rod 206.

Continuing to block 506 “send, after the selected time period, a control signal to a shutter to open the shutter” the shutter 124 can be opened after the selected time period (e.g., the “warm up” period). For example, processing circuitry of controller 104 can execute instructions stored in memory to cause controller 104 to send, after the warmup time period, a control signal to the shutter 124 to cause the shutter 124 to open. It is assumed that the shutter 124 is closed at the initiation of routine 500 (e.g., as the medical laser system 100 is inactive). However, routine 500 could be provided with a block to close the shutter at the start of routine 500.

Continuing to block 508 “activate the optical pump source at a repetition rate and output energy specified for the treatment” the optical pump source 204 can be activated at a repetition rate and output energy specified for the treatment. For example, in some embodiments, controller 104 can be configured to receive indications (e.g., via display 106, or the like) of the treatment (e.g., stone dusting, tissue ablation, or the like) and/or frequencies and powers for the pulsed laser beam 216. Processing circuitry of controller 104 can execute instructions stored in memory to cause controller 104 to send a control signal to optical pump source 204 to cause optical pump source 204 to energize at a repetition rate and power associated with the treatment.

Accordingly, routine 500 provides that the laser rod 206 can be warmed up to a steady state temperature without the risk of damaging the rod. That is, as any emissions resulting from the energies of optical pump source 204 at block 504 will have negligible energies as they are generated proximate to the lasing threshold.

Further, at the warmup process can be completed in an insignificant amount of time (e.g., 200 ms, or the like); the delay between activation of the input device (e.g., depression of the pedal, or the like) and the first pulse is also insignificant.

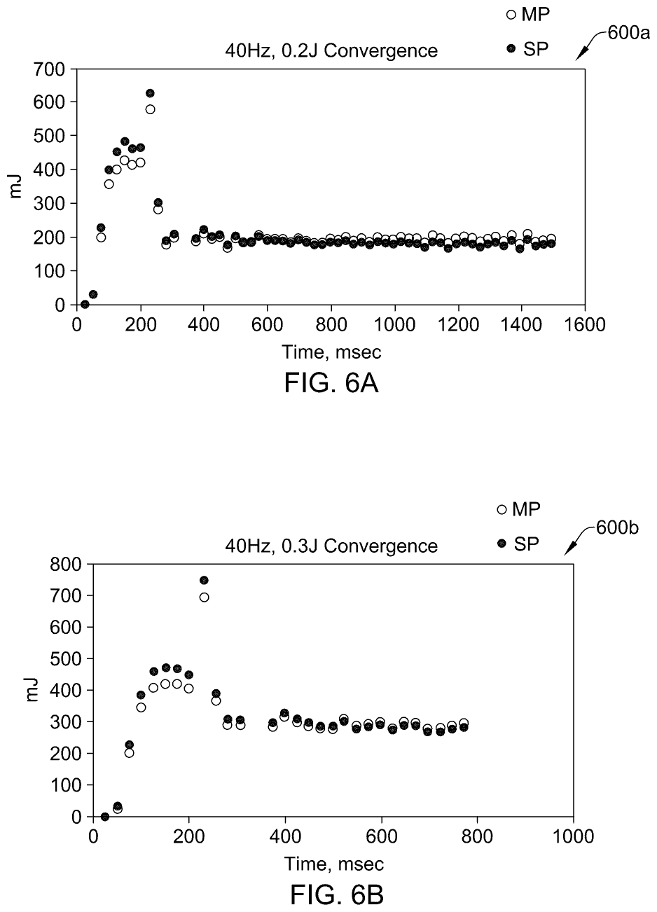

FIG. 6A and FIG. 6B illustrate graphs 600a and 600b, respectively, which depict the convergence of output power in millijoules (mJ) on the y-axis versus time in ms on the x-axis. Both graphs represent a pulsed output at 40 Hz.

For example, FIG. 6A illustrates graph 600a showing convergence at 0.2 mJ, which takes ˜200 ms to converge.

Similarly, FIG. 6B illustrates graph 600b showing convergence at 0.3 mJ, which takes ˜200 ms to converge.

Importantly, FIG. 6A and FIG. 6B illustrate that only a few initial pulses exceed the specified energy.

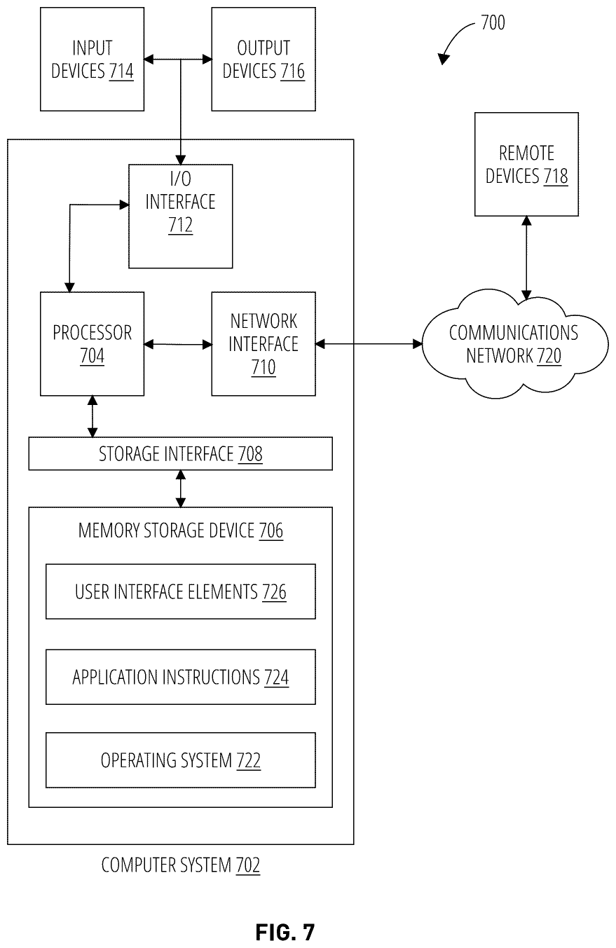

FIG. 7 is a block diagram of a computing environment 700 including a computer system 702 for implementing embodiments consistent with the present disclosure. In some embodiments, the computing environment 700, or portion thereof (e.g., the computer system 702) may comprise or be comprised in a laser system (e.g., the controller 104 of the medical laser system 100 can embody portions of the computing environment 700). Accordingly, in various embodiments, computer system 702 may be configured to control activation of an optical pump source 204 and shutter 124 as outlined herein.

The computer system 702 may include a central processing unit (“CPU” or “processor”) 704. The processor 704 may include at least one data processor for executing instructions and/or program components for executing user or system-generated processes. A user may include a person, a person using a device such as those included in this disclosure, or another device. The processor 704 may include specialized processing units such as integrated system (bus) controllers, memory management control units, floating point units, graphics processing units, neural processing units, digital signal processing units, etc. The processor 704 may be disposed in communication with input devices 714 and output devices 716 via I/O interface 712. The I/O interface 712 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 712, computer system 702 may communicate with input devices 714 and output devices 716. In some embodiments, the processor 704 may be disposed in communication with a communications network 720 via a network interface 710. In various embodiments, the communications network 720 may be utilized to communicate with a remote memory storage device 706, such as for accessing look-up tables, performing updates, or utilizing external resources. The network interface 710 may communicate with the communications network 720. The network interface 710 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 720 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 720 may include a variety of network devices, including routers, bridges, servers, computing devices, storage devices, etcetera. In some embodiments, the processor 704 may be disposed in communication with a memory storage device 706 via a storage interface 708. The storage interface 708 may connect to memory storage device 706 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 706 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 706 may store a collection of program or database components, including, without limitation, an operating system 722, an application instructions 724, and a user interface elements 726. In various embodiments, the operating system 722 may facilitate resource management and operation of the computer system 702. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM® OS/2®, MICROSOFT® WINDOWS® (XP®, VISTA®/7/8, 10 etc.), APPLE® IOS®, GOOGLE™ ANDROID™, BLACKBERRY® OS, or the like.

What is claimed is:

1. An optical resonator for a medical laser system, comprising:
a housing;
an optical pump source disposed in the housing;
an insert disposed in the housing;
a lasing medium disposed in the insert;
a high-reflectivity (HR) mirror disposed outside the housing a first distance away from a first end of the lasing medium; and
an output coupler (OC) mirror disposed outside the housing a second distance away from a second end of the lasing medium,
wherein the first end of the lasing medium is opposite the second end of the lasing medium, and
wherein the first distance is substantially equal to the second distance.

2. The optical resonator of claim 1, wherein the HR mirror, the OC mirror, or both the HR mirror and the OC mirror are flat mirrors.

3. The optical resonator of claim 1, wherein the insert comprises Samarium (Sm).

4. The optical resonator of claim 3, wherein the insert is Sm-doped Fused Silica.

5. The optical resonator of claim 1, wherein the first distance and the second distance are greater than or equal to 0 millimeters (mm) and less than or equal to 120 mm.

6. The optical resonator of claim 1, wherein the lasing medium is a Ho:YAG lasing rod or a CTH:YAG lasing rod.

7. The optical resonator of claim 1, wherein the optical pump source is configured to energize at a frequency of greater than or equal to 20 Hertz (Hz).

8. The optical resonator of claim 7, wherein the optical pump source is configured to energize at a frequency of greater than 20 Hz and less than or equal to 45 Hz.

9. A method for energizing an optical resonator for a medical laser system, comprising:
receiving an indication to initiate lasing by the optical resonator;
activating, for a selected time period at a preselected repetition rate and output energy, an optical pump source of the optical resonator;
sending, after a selected delay, a control signal to a shutter of the medical laser system to cause the shutter to open; and
activating the optical pump source at a repetition rate and output energy specified for a treatment,
wherein the preselected repetition rate is greater than or equal to 85 percent of a maximum repetition rate of the optical pump source, and
wherein the preselected output energy is substantially equal to a lasing threshold of a laser medium of the optical resonator.

10. The method of claim 9, wherein the selected delay equals the selected time period.

11. The method of claim 10, wherein the selected delay and the selected time period are less than or equal to 200 milliseconds (ms).

12. The method of claim 9, wherein the maximum repetition rate of the optical pump source is greater than or equal to 20 Hertz (Hz).

13. The method of claim 12, wherein the maximum repetition rate of the optical pump source is greater than 20 Hz and less than or equal to 45 Hz.

14. The method of claim 9, wherein the optical resonator comprises:
a housing,
the optical pump source disposed in the housing;
an insert disposed in the housing;
the lasing medium disposed in the insert;
a high-reflectivity (HR) mirror disposed outside the housing a first distance away from a first end of the lasing medium; and
an output coupler (OC) mirror disposed outside the housing a second distance away from a second end of the lasing medium,
wherein the first end of the lasing medium is opposite the second end of the lasing medium, and
wherein the first distance is substantially equal to the second distance.

15. The method of claim 14, wherein the HR mirror, the OC mirror, or both the HR mirror and the OC mirror are flat mirrors.

16. The method of claim 14, wherein the insert comprises Samarium (Sm).

17. The method of claim 14, wherein the insert is Sm-doped Fused Silica.

18. The method of claim 14, wherein the lasing medium is a Ho:YAG lasing rod or a CTH:YAG lasing rod.

19. A medical laser system, comprising:
at least one optical resonator, each of the at least one optical resonators comprising:
a housing,
an optical pump source disposed in the housing,
an insert disposed in the housing,
a lasing medium disposed in the insert,
a high-reflectivity (HR) mirror disposed outside the housing a first distance away from a first end of the lasing medium, and
an output coupler (OC) mirror disposed outside the housing a second distance away from a second end of the lasing medium;
a shutter;
at least one mirror configured to reflect an output emission from the at least one optical resonator to the shutter; and
a coupling assembly comprising at least one lens configured to optically couple the output emission with a proximal end of an optical fiber,
wherein the first end of the lasing medium is opposite the second end of the lasing medium, and
wherein the first distance is substantially equal to the second distance.

20. The medical laser system of claim 19, wherein the insert is Sm-doped Fused Silica.
